(19) United States
(12) Patent Application Publication
(10) Pub. No.: US 2005/0055051 A1
(43) Pub. Date: Mar. 10, 2005

(54) High strength suture with silk trace

(76) Inventor: R. Donald Grafton, Naples, FL (US)

Correspondence Address: DICKSTEIN SHAPIRO MORIN & OSHINSKY LLP, 2101 L Street, NW, Washington, DC, 20037, US

(21) Appl. No.: 10/970,381

(22) Filed: Oct. 22, 2004

Related U.S. Application Data
(63) Continuation-in-part of application No. 10/358,399, filed on Feb. 5, 2003.
Application No. 10/358,399 is a continuation-in-part of application No. 10/160,176, filed on Jun. 4, 2002.
Application No. 10/358,399 is a continuation-in-part of application No. 09/950,598, filed on Sep. 13, 2001, now Pat. No. 6,716,234.
(60) Provisional application No. 60/354,499, filed on Feb. 8, 2002.
(60) Provisional application No. 60/350,040, filed on Jan. 23, 2002.
(60) Provisional application No. 60/330,913, filed on Nov. 2, 2001.

(51) Int. Cl. A61B 17/04

(52) U.S. Cl. 606/228

(57) ABSTRACT

A high strength abrasion resistant surgical suture material with improved tie down characteristics and tissue compliance is color coded for visualization and identification purposes. The suture features a multifilament jacket formed of braided strands of ultra high molecular weight polyethylene and polyester, with silk fibers included in a color contrasting with the other jacket fibers to provide an identifiable trace. In one embodiment, the braided jacket surrounds a core formed of twisted strands of ultrahigh molecular weight polyethylene. The suture, provided in a No. 2 size, has the strength of No. 5 Ethibond, is ideally suited for most orthopedic procedures, and can be attached to a suture anchor or a curved needle. The identifiable silk trace preferably may be provided along one half of the length of the suture, so that when the suture is loaded onto a suture anchor, for example, the two legs of the length of suture on either side of the suture anchor can be readily distinguished.

### CROSS-REFERENCE TO RELATED APPLICATIONS

This application is a continuation-in-part of U.S. application Ser. No. 10/358,399, filed Feb. 5, 2003, which is a continuation-in-part of U.S. application Ser. No. 10/160,176, filed Jun. 4, 2002, which claims the benefit of U.S. Provisional Application No. 60/354,499, filed Feb. 8, 2002, U.S. Provisional Application No. 60/350,040, filed Jan. 23, 2002 and U.S. Provisional Application No. 60/330,913, filed Nov. 2, 2001, which is a continuation-in-part of U.S. application Ser. No. 09/950,598, filed Sep. 13, 2001, now U.S. Pat. No. 6,716,234.

### BACKGROUND OF THE INVENTION

1. Field of the Invention

The present invention relates to high strength surgical suture materials, and more particularly to braided suture blends of ultrahigh molecular weight polyethylene having tinted silk strands.

2. Description of the Related Art

Suture strength is an important consideration in any surgical suture material. One of the strongest materials currently formed into elongated strands is an ultrahigh molecular weight long chain polyethylene, typically used for fishing line and the like, which is sold under the trade names Dyneema or Spectra. This material is much stronger than ordinary surgical suture, however, it does not have acceptable knot tie down characteristics for use in surgical applications.

### BRIEF SUMMARY OF THE INVENTION

The present invention advantageously provides a high strength surgical suture material with improved tie down characteristics. The suture features a braided jacket made of ultrahigh molecular weight fibers and silk fibers. The polyethylene provides strength. Polyester fibers made be woven with the high molecular weight weight polyethylene to provide improved tie down properties. The silk is provided in a contrasting color to provide a trace for improved suture recognition and identification. Silk also is more tissue compliant than other fibers, allowing the ends to be cut close to the knot without concern for deleterious interaction between the ends of the suture and surrounding tissue. Handling properties of the high strength suture also can be enhanced using various materials to coat the suture.

Strands of a contrasting color added to the braided threads make the suture more discernable during surgical procedures. The colored strands preferably are dyed filaments of natural silk. In one embodiment, half of a length of suture is provided with tinted tracing strands, or otherwise contrasts visually with the other half of the length of suture, which remains a plain, solid color, or displays a different braiding pattern, for example. Accordingly, when the length of suture is loaded through the eyelet of a suture anchor or passed through tissue, for example, at least one of the legs of the suture is visually coded, making identification and handling of the suture legs simpler, particularly during arthroscopic surgery.

In a preferred embodiment, the suture includes a multifilament jacket formed of ultrahigh molecular weight polyethylene fiber braided with polyester and colored silk fibers. The jacket surrounds a fiber core made substantially or entirely of ultrahigh molecular weight polyethylene. The core preferably includes three strands of ultrahigh molecular weight polyethylene, twisted at about three to six twists per inch.

The jacket preferably comprises eight strands of ultrahigh molecular weight polyethylene braided with six strands of polyester and one or two strands of a tinted silk fiber. The tinted silk strands can be included in black or some other contrasting color as explained in greater detail below.

Ultrahigh molecular weight polyethylene fibers suitable for use in the present invention are marketed under the Dyneema trademark by Toyo Boseki Kabushiki Kaisha, and are produced in the U.S. by Honeywell under the trademark Spectra.

The suture of the present invention advantageously has the strength of Ethibond No. 5 suture, yet has the diameter, feel and tie-ability of No. 2 suture. As a result, the suture of the present invention is ideal for most orthopedic procedures such as rotator cuff repair, Achilles tendon repair, patellar tendon repair, ACL/PCL reconstruction, hip and shoulder reconstruction procedures, and replacement for suture used in or with suture anchors.

The suture can be uncoated, or coated with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the braid, knot security, or abrasion resistance, for example.

The dyed silk fibers in the jacket are provided in a contrasting color for visibility and identification purposes. A trace thread or two in the suture jacket aids surgeons in identifying the travel direction of the suture during surgery, particularly during operations viewed arthroscopically or remotely. Providing the trace threads in a regularly repeating pattern is particularly useful, allowing the surgeon to decode different ends of a length of suture, and to determine the direction of travel of a moving length of suture. The trace threads preferably are provided uniquely on each half of a length of suture to allow for tracing and identification of each end of the suture, such as when the suture is threaded through an eyelet of a suture anchor. Silk is a strong natural fiber that can be dyed.

Other features and advantages of the present invention will become apparent from the following description of the invention which refers to the accompanying drawings.

### BRIEF DESCRIPTION OF THE DRAWINGS

FIG. 1 is a copy of a scanning electron micrograph of a length of suture according to the present invention.

FIG. 2 is a schematic cross section of a length of suture according to the present invention.

FIG. 3 is an illustration of the suture of the present invention attached to a suture anchor loaded onto a driver.

FIGS. 4A and 4B show the suture of the present invention attached to a half round, tapered needle.

FIG. 5 illustrates a bulk length of suture of the present invention.

FIG. 6 illustrates a strand of suture according to the present invention provided on a suture anchor.

### DETAILED DESCRIPTION OF THE INVENTION

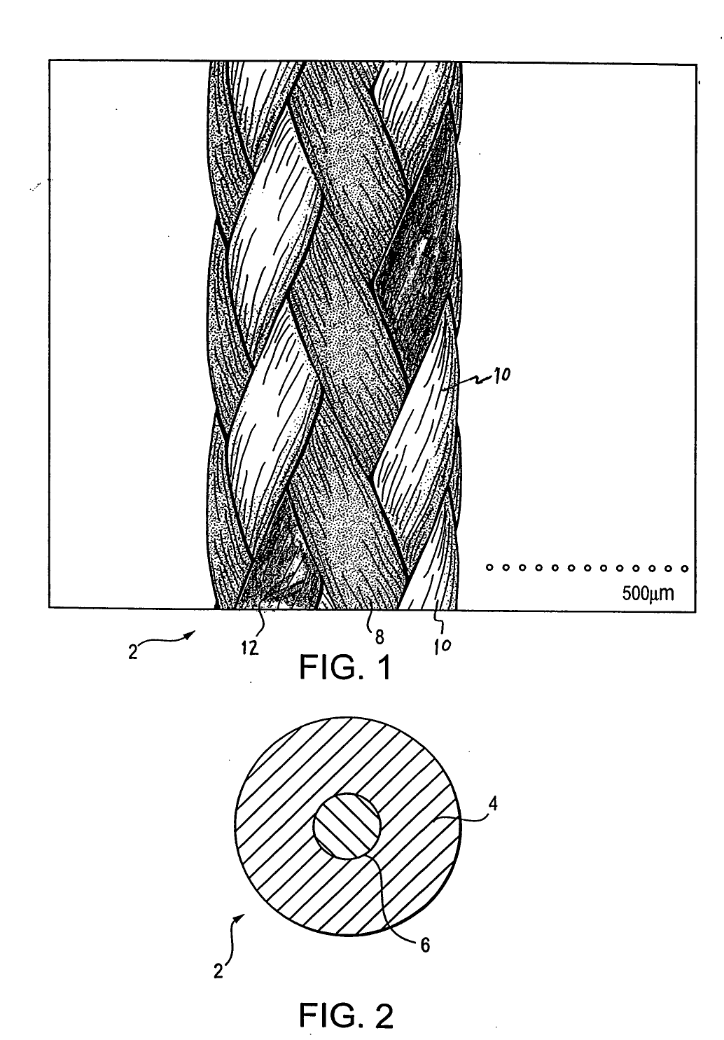

Referring to FIG. 1, a scanning electron micrograph of a length of suture 2 according to the present invention is shown. Suture 2 is made up of a jacket 4 and a core 6 surrounded by the jacket 4. See FIG. 2. Strands of ultrahigh molecular weight polyethylene (UHMWPE) 8, such as that sold under the tradenames Spectra and Dyneema, strands of polyester 10, and tinted strands 12 are braided together to form the jacket 4. Core 6 is formed of twisted strands of UHMWPE.

UHMWPE, used for strands 8, is substantially translucent or colorless. The polyester strands 10 are white (undyed). Tinted silk strands 12 are tinted a contrasting color to provide a visible trace in the suture. Due to the transparent nature of the UHMWPE, the suture takes on the color of strands 10 and 12, and thus appears to be white with a trace in the contrasting color.

In accordance with the present invention, silk traces 12 preferably are black. The black trace assists surgeons in distinguishing between suture lengths with the trace and suture lengths without the trace. Traces also assist the surgeon in identifying whether the suture is moving. The trace can extend the entire length of the suture or only on half of a length of suture, the other half of the suture length remaining plain (white). Alternatively, the traces can form visibly distinct coding patterns on each half of the suture length. As a result, when the suture is threaded through the eyelet of a suture anchor, for example, the two legs (halves) of the length of suture are easily distinguished, and their direction of travel will be readily evident when the suture is pulled during surgery.

Details of the present invention will be described further below in connection with the following examples:

### EXAMPLE 1

### USP Size 5 (EP Size 7)

Made on a 16 carrier Hobourns machine, the yarns used in the braided jacket are Honeywell Spectra 2000, polyester type 712, and nylon. The jacket is formed using eight strands of 144 decitex Spectra per carrier, braided with six strands of 100 decitex polyester, and two strands of tinted nylon. The core is formed of three carriers of 144 decitex Spectra braided at three to six twists per inch. A No. 5 suture is produced.

### EXAMPLE 2

### Silk-Size 1

| | |
|---|---|
| Core: | 1 end of 144 dtex Spectra × 3 |
| Jacket: | 5 carriers 95 dtex polyester |
| | 6 carriers 144 dtex Spectra |
| | 1 carrier 84 dtex silk |

### EXAMPLE 3

### Silk-Size 2

| | |
|---|---|
| Core: | 1 end of 144 dtex Spectra × 3 |
| Jacket: | 5 carriers 95 dtex polyester |
| | 8 carriers 144 dtex Spectra |
| | 1 carrier 84 dtex silk |

To make various sizes of the inventive suture, different decitex values and different PPI settings can be used to achieve the required size and strength needed. In addition, smaller sizes may require manufacture on 12 carrier machines, for example. The very smallest sizes can be made without a core. Overall, the suture may range from 5% to 90% ultrahigh molecular weight polymer (preferably at least 40% of the fibers are ultrahigh molecular weight polymer), with the balance formed of polyester and silk. The core preferably comprises 18% or greater of the total amount of filament.

The suture preferably is coated with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the braid, knot security, or abrasion resistance, for example.

The ultra high molecular weight (UHMW) polymer component of the present invention provides strength, and the polyester component is provided to improve tie ability and tie down characteristics. However, it has been found that the UHMW polymer provides an unexpected advantage of acting as a cushion for the polyester fibers, which are relatively hard and tend to damage each other. The UHMW polymer prevents breakage by reducing damage to the polyester when the suture is subjected to stress.

According to an alternative embodiment of the present invention, a partially bioabsorbable suture is provided by blending a high strength material, such as UHMVVPE fibers, with a bioabsorbable material, such as PLLA or one of the other polylactides, for example. For example, a suture made with about 10% Spectra or Dyneema blended with absorbable fibers would provide greater strength than existing bioabsorbable suture, and with less stretch. Over time, 90% or more of the suture would absorb, leaving only a very small remnant of the knot. The absorbable suture can include coatings and tinted traces as noted above for nonabsorbable suture.

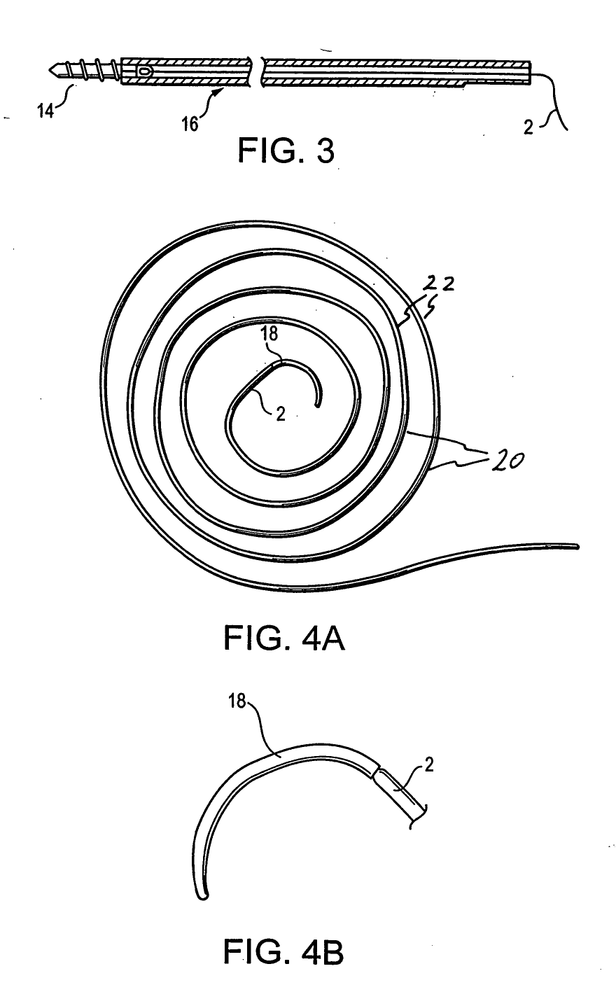

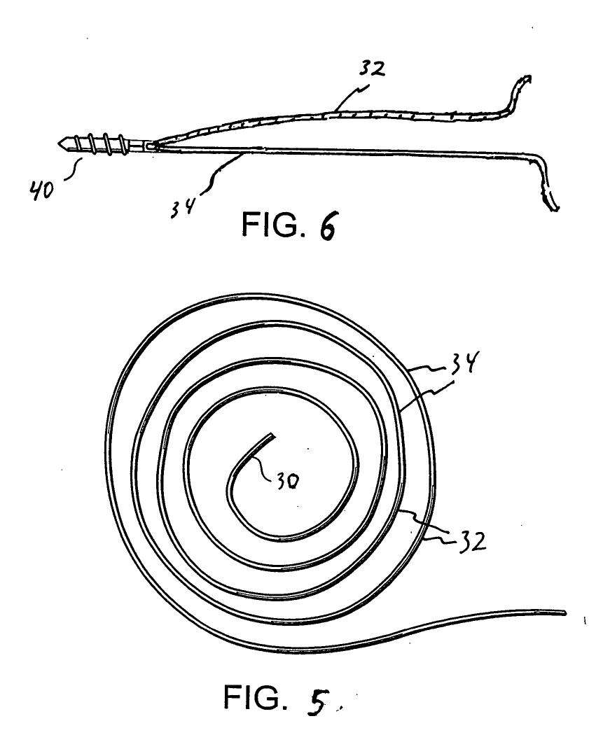

In one method of using the suture of the present invention, the suture 2 is attached to a suture anchor 14 as shown in FIG. 3 (prepackaged sterile with an inserter 16), or is attached at one or both ends to a half round, tapered needle 18 as shown in FIGS. 4A and 4B. FIG. 4A also illustrates a length of suture having regularly repeating pattern of trace threads according to the present invention. Sections 20 of the length of suture 2 have tinted tracing threads woven in, where sections 22 of the length of suture are plain, or otherwise are distinguishable from sections 20. The alternating patterned and plain sections aid the surgeon in determining the direction of suture travel when pulling the suture, for example.

As shown in FIG. 5, to make the suture which has a trace only at one end, bulk suture 30 is provided with repeating sections 32 having trace threads separated by sections 34 having no trace threads. The bulk suture is cut between every other section, at one end of each plain section, for example, to provide lengths of suture that are half traced and half plain. Alternatively, the bulk suture can be cut midway through each section to provide a shorter suture having a trace at one end. The half-and-half lengths of suture can be threaded through the eyelet of a suture anchor 40, as shown in FIG. 6. Accordingly, the identity of each leg of the suture strand provided on the suture anchor is easily decoded by a surgeon operating with the suture anchor assembly.

One or more strands of silk in the blend are be provided in pre-dyed colors, e.g., black, to provide a trace. For additional variety, different patterns can be used to incorporate the tinted trace into the suture. The trace threads enhance the ability to visually detect suture motion and to differentiate between suture strands.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

1. A suture strand suitable for use as a suture or ligature comprising a plurality of braided fibers of ultrahigh molecular weight polyethylene and at least one visually contrasting silk fiber, the visually contrasting silk fiber providing a visually distinguishable trace along at least a portion of the suture strand.

2. The suture strand of claim 1, having the visually distinguishable trace on one end portion and a plain section with no identifiable trace or a different identifiable trace on an opposite end portion.

3. The suture strand of claim 2, wherein the strand includes only two sections, the plain section and a section with the identifiable trace.

4. The suture strand of claim 1, further comprising a plurality of fibers of at least one long chain synthetic polymer or bioabsorbable fiber braided with the ultrahigh molecular weight polyethylene.

5. The suture strand of claim 4, wherein the at least one long chain synthetic polymer is polyester, nylon, or both.

6. The suture strand of claim 4, wherein the ultrahigh molecular weight polyethylene comprises at least 40% of the braided fibers.

7. The suture strand of claim 4, wherein the polyester comprises less than about 40% of the braided filaments.

8. The suture strand of claim 1, further comprising a core of twisted fibers of ultrahigh molecular weight polyethylene surrounded by a jacket comprising the plurality of braided fibers of ultrahigh molecular weight polyethylene and polyester.

9. The suture strand of claim 8, wherein the core comprises about 18% or greater of the total amount of filament.

10. The suture strand of claim 8, wherein the jacket comprises less than about 82% of the total amount of filament.

11. The suture strand of claim 8, further comprising a coating disposed on the jacket.

12. The suture strand of claim 11, wherein the coating is selected from the group consisting of wax, silicone, silicone rubbers, PTFE, PBA, and ethyl cellulose.

13. A suture assembly comprising:
a suture formed of a plurality of braided fibers of ultrahigh molecular weight polyethylene and at least one silk fiber visually contrasting with the remaining fibers along at least a portion of the suture; and
a suture anchor attached to the suture.

14. The suture assembly as recited in claim 13, further comprising polyester fibers.

15. A suture assembly comprising:
a suture formed of a plurality of braided fibers of ultrahigh molecular weight polyethylene and at least one silk fiber visually contrasting with the remaining fibers along at least a portion of the suture; and
a half round, tapered needle attached to one or both ends of the suture.

16. A suture strand suitable for use as a suture or ligature including an outer jacket comprising a plurality of braided fibers of ultrahigh molecular weight polyethylene and at least one silk fiber which is tinted to provide an identifiable trace along at least a portion of the length of the suture strand.
